(19) United States
(12) Patent Application Publication
(10) Pub. No.: US 2011/0054445 A1
(43) Pub. Date: Mar. 3, 2011

(54) Devices and Methods for Treatment of Eye Disease

(75) Inventors: Henry Rylander, Round Rock, TX (US); Ashley J. Welch, Burnet, TX (US); Raiyan Zaman, Austin, TX (US)

(73) Assignee: BOARD OF REGENTS, THE UNIVERSITY OF TEXAS SYSTEM, Austin, TX (US)

(21) Appl. No.: 12/874,851

(22) Filed: Sep. 2, 2010

Related U.S. Application Data
(60) Provisional application No. 61/239,341, filed on Sep. 2, 2009.

(51) Int. Cl. A61M 31/00; A61B 18/18

(52) U.S. Cl. 604/521; 606/4

(57) ABSTRACT

Methods, systems, and apparatus for delivering drugs for treatment of eye disease are described. For example, the methods, systems, and apparatus can be optionally used to treat wet age-related macular degeneration. In some examples, an eye disease is treated by implanting a device comprising a sealed container into the diseased eye. A dosage of drug for treating the diseased eye is disposed within the sealed container. The dosage of the drug is selectively released from the sealed container of the implanted device into eye tissue. The released dosage treats the eye disease.

### CROSS-REFERENCE TO RELATED APPLICATIONS

This application claims the benefit of U.S. Provisional Patent Application No. 61/239,341, filed Sep. 2, 2009, which is incorporated herein by reference in its entirety.

### STATEMENT REGARDING FEDERALLY SPONSORED RESEARCH

This invention was made with government support under Grant No. BES-0529340 awarded by the National Science Foundation. The government has certain rights in the invention.

### BACKGROUND

Treatment of many eye diseases, such as age-related macular degeneration, infections, diabetes and uveitis, may require multiple intraocular injections of one or more therapeutic agent, Multiple injections of therapeutic agents can be painful, increase the risk of endopthalmitis, and cause a host of other adverse side effects.

### SUMMARY

Provided herein are devices and methods related to eye disease treatment. For example, the methods and devices can be used to treat wet age-related macular degeneration and other eye diseases.

In one aspect, a provided method includes implanting a device comprising a sealed container into the diseased eye. A dosage of a drug for treating the diseased eye is disposed within the sealed container. The method also includes selectively releasing the dosage of drug from the sealed container of the implanted device into eye tissue. The released dosage treats the eye disease.

This, and other aspects, can include one or more of the following features. The device can be implanted under the sclera of the eye. For example, the device can be implanted in the superchoroidal space of the eye. The device can also be implanted in the posterior chamber of the eye. Light can be directed onto the sealed container of the implanted device. The light can be adjusted to rupture at least a portion of the sealed chamber such that the drug dosage is selectively released from the chamber into eye tissue. Light can be directed through the sclera and then onto the sealed container of the implanted device. The light can cause rupture of at least a portion of the sealed chamber such that the drug dosage is selectively released from the chamber into eye tissue.

The method can include treating the sclera with an agent that alters the optical properties of the sclera to allow increased transmission of light through the sclera. The scleral treatment can occur prior to directing the light through the sclera and onto the sealed container of the implanted device. The disease can be wet age-related macular degeneration. The implanted device can include multiple sealed containers. A dosage of drug for treating the diseased eye can be disposed within each sealed container. Each sealed container of the implanted device can be configured to be individually ruptured to allow drug from the individually ruptured sealed container to enter the eye tissue. A first sealed container can be ruptured, and a second sealed container can be ruptured at a predetermined time subsequent to the rupture of the first sealed container.

According to another aspect, methods for delivering a drug include treating a region adjacent to a biological tissue with an agent configured to render the region at least partially transparent to light or more transparent to light if the tissue is at least partially transparent to light. A drug delivery device including multiple reservoirs is positioned in the region. Each reservoir includes a drug. Optionally, each reservoir contains the same drug. Optionally, at least two reservoirs contain different drugs. The method includes rupturing the drug delivery device by applying an energy generated from an energy source, on the drug delivery device. The energy has an energy level sufficient to rupture the drug delivery device.

This, and other aspects, can include one or more of the following features. The energy can be optical energy. The method can further include generating an optical beam from a laser source, and manipulating the optical beam to be incident on a reservoir including the drug. Optionally the optical beam is pulsed and the optical source can deliver one or more optical pulses of laser light to be incident on a reservoir including the drug.

The energy level of the optical beam can rupture the reservoir on which the optical beam is incident. The region adjacent to the biological tissue can be in an eye. The method can further include implanting the drug delivery device in the eye. An agent can be used to render the region at least partially transparent to permit an optical beam from a Q-switched Nd-YAG laser source (or similar pulsed laser) to pass through a surface of the eye and on the drug delivery device. For example, any Q-switched laser operating in a stress confinement mode can be used. For example, a Q-switched Ho-YAG laser with a pulse duration of 10 nsec can be used to disrupt a seal/membrane of an implanted device. The drug delivery device can include a leading edge suture and a trailing edge suture attached to a leading edge and a trailing edge, respectively, of the drug delivery device. Implanting the drug delivery device in the eye can include surgically attaching the drug delivery device in the eye with the leading edge suture and the trailing edge suture.

In another aspect, methods of delivering a drug include implanting a drug delivery device in a region of a biological tissue. Energy from an energy source is applicable to the region. The drug delivery device includes a reservoir containing a drug. The reservoir is configured to rupture when exposed to the energy thereby releasing the drug. The method includes applying energy from the energy source to the reservoir of the drug delivery device. The drug in the reservoir is released when the reservoir or a portion thereof, exposed to the energy, ruptures.

This, and other aspects can include one or more of the following features. The energy source can be a Q-switched Nd-YAG laser. The method can further include treating the region with an agent configured to render the region at least partially transparent to light. Applying the energy from the energy source can include transmitting an optical beam generated by the Q-switched Nd-YAG laser through the at least partially transparent region. The region of the biological tissue in which the drug delivery device is implanted can be in the eye.

In another innovative aspect, an apparatus to deliver a drug is provided. The apparatus includes a device that includes multiple reservoirs and a drug to be delivered. A dosage of the drug is included in each reservoir of the multiple reservoirs. The apparatus includes a seal sealing the multiple reservoirs. The seal includes a material which ruptures when an optical beam from a laser source is incident on the seal.

This, and other aspects, can include one or more of the following features. One or more reservoirs can be 2.6 mm wide. The device including the seal can be 1.501 mm in height. The seal can be 0.01 mm thick. Each reservoir can be positioned at a distance of 0.3 mm from an edge of the device. A portion of each reservoir can be formed by the seal. The device can be 6 mm wide.

Also provided is an apparatus for use in treating an eye disease. The apparatus includes an ocular implant including multiple sealed containers. Each sealed container holds a dosage of drug for treating the eye disease. Each sealed container of the implanted device is configured for selective release of its dosage of drug into tissue of the eye to treat the eye disease.

This, and other aspects, can include one or more of the following features. The ocular implant is configured for positioning beneath the sclera of the eye to be treated. For example, the ocular implant can be configured for positioning in the superchoroidal space of the eye to be treated. In another example, the ocular implant can be configured for positioning in the posterior chamber of the eye to be treated. At least a portion of one or more of the sealed containers can be configured to be ruptured when contacted by light. The rupturing can allow for selective release of the ruptured container's dosage of drug into eye tissue. One or more of the sealed containers can hold a dosage of ranibizumab or another drug/therapeutic agent.

Devices (for example, apparatuses), and methods described in this specification can provide a patient with the option of a one time implantation of a carrier system containing multiple doses of the drug followed by controlled release of individual doses of the drug in the apparatus using a Q-switched Nd-YAG laser or another laser as described herein. This can eliminate the need for multiple painful injections, thereby reducing patient discomfort, and can decrease the possibility of contracting infections. Further, because the drug that is carried in the apparatus can be released using a laser, the drug delivery can be accomplished using non-invasive techniques. The same apparatus can be used for multiple treatments by rupturing one of many reservoirs fabricated into the apparatus. By controlling the dosage of the drug in each reservoir, the therapeutic efficacy of the drug can be improved. Furthermore, using the drug delivery device described here, precise and repeatable doses of medication can be provided to patients.

The details of one or more implementations of the specification are set forth in the accompanying drawings and the description below. Other features, aspects, and advantages of the specification will become apparent from the description, the drawings, and the claims.

### BRIEF DESCRIPTION OF THE DRAWINGS

FIG. 1 is a schematic illustration of an example system for delivering a drug.

FIG. 2 is a schematic illustration showing an example method to deliver a drug.

FIG. 3 is a schematic illustration showing an example apparatus to deliver a drug.

FIG. 4 is a photograph showing an example drug delivery device.

FIG. 5 is a photograph showing a reservoir ruptured using an optical beam.

Like reference numbers and designations in the various drawings indicate like elements.

### DETAILED DESCRIPTION

A biocompatible drug delivery device for treating an eye disease is described. In some implementations, the apparatus includes an ocular implant that includes multiple sealed containers, each of which holds a dosage of drug for treating the eye disease. As described with reference to the following figures, an example implanted device is configured for selective release of its dosage of drug into eye tissue to treat the eye disease.

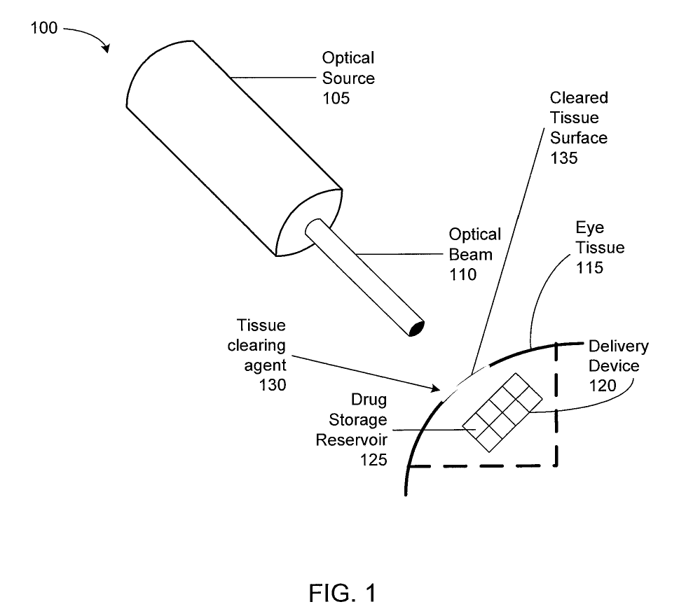

An example of a system that includes such a drug delivery device is described with reference to FIG. 1. FIG. 1 shows an example of a system 100 for delivering a drug. The system includes a delivery device 120 that includes multiple drug storage reservoirs 125. Each reservoir 125 includes a dosage, for example, a dosage of a drug indicated for the treatment of the diseased eye. One or more reservoir can optionally contain soluble drugs/therapeutic agents that are packaged as liquids. One or more reservoir can optionally contain one or more encapsulated solid drug pellets.

In some implementations, the delivery device 120 is implanted beneath a surface of the eye tissue 115, for example, under the sclera of the eye. For example, the device can be implanted in the super-choroidal or subscleral space of the eye. In another example, the device can be implanted in the posterior chamber of the eye. In another example, the device can be implanted in a secleral pocket formed within the sclera. In another example, the device can be implanted episclerally, for example within the conjunctiva.

To deliver the drug in each reservoir 125, the reservoir 125 can be selectively ruptured by applying energy generated from an energy source. In some implementations, the energy source is an optical source 105, for example, a Q-switched Nd-YAG laser configured to generate an optical beam 110, for example, an Nd-YAG laser beam. The optical source can provide one or more pulses of laser light (e.g. one or more optical beam pulses) which contact the delivery device 120. Optionally, a pulse of laser light may contact the seal of one or more reservoir.

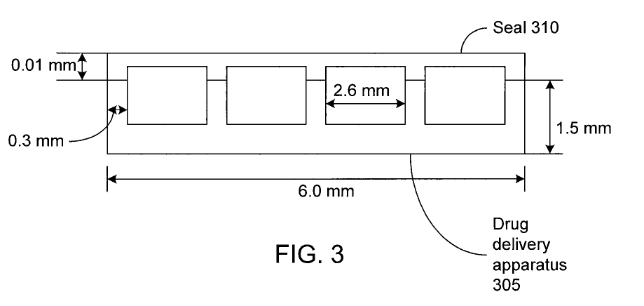

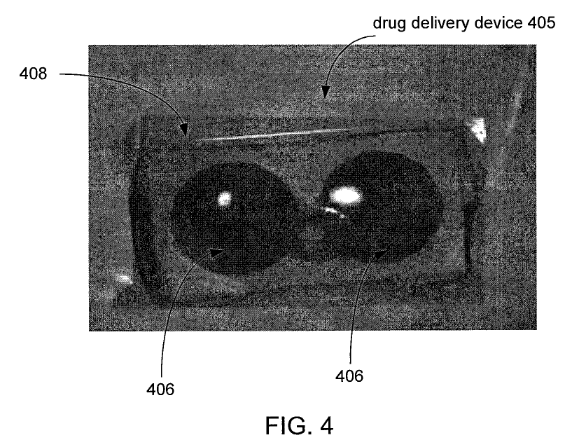

A seal 310 is shown schematically in FIG. 3 and a seal 408 is shown in FIG. 4. One or more pulses of light can rupture the seal allowing for release of drug contained in the reservoir without substantially heating or melting the seal. Thus the seal can be ruptured without melting the seal with heat from a laser source. When a pulsed laser source is used, the one or more pulse can create a shock wave that disrupts the seal/membrane rather than melting it. A pulsed laser that can be used to open the reservoir (e.g. by disruption of the seal/membrane) without melting any portion of the device is a Q-switched Nd-YAG laser source, or a similar laser source, operating in a stress confinement mode. Optional operating parameters for such a laser are an energy of about 15 mJ, a pulse time of 10 nsec, and a beam diameter of about 100 microns.

The delivery device 120 is fabricated using a material that is biocompatible. At least a part of the delivery device 120 is capable of rupturing when the energy from the energy source has an energy level that is greater than a threshold which can rupture the device. For example, when the power of the optical beam 110 generated by the optical source 105 and incident on a reservoir 125 is greater than a threshold power, the material of the delivery device 120 ruptures, releasing the drug stored in the reservoir 125 into the region beneath the eye tissue 115 where the device 120 is implanted. In some implementations, the power of the optical beam 110 is between and including 6.5 mJ and 10.5 mJ. Because the device 120 is implanted in or proximate a diseased region of the eye, the drug spreads through the diseased region by diffusion thereby treating the disease.

The methods and agents as described herein are useful for therapeutic treatment. Therapeutic treatment involves administering to a subject a therapeutically effective amount of the agents/drugs described herein. The terms effective amount and effective dosage are used interchangeably. The term effective amount is defined as any amount necessary to produce a desired physiologic response (e.g., a reduction in symptoms associated with macular degeneration). Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage may vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein, the terms treatment, treat, or treating refers to a method of reducing the effects of a disease or condition (e.g., macular degeneration) or symptom of the disease or condition. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

The described methods and devices can be used to treat eye disease and can reduce or eliminate the need for multiple injections of therapeutic agent into a patient's eye. For example, age-related macular degeneration is a chronic eye disease in which the maculae gradually deteriorate causing blurred central vision or a blind spot in the center of the visual field. A current treatment for wet macular degeneration is periodic injection of a drug such as Lucentis® (Ranibizumab injection), Genentech Inc. (San Francisco, Calif.), into the eye. This form of treatment, which often requires multiple injections of the drug into the eyes of patients, can be painful and can increase the risk of endophthalmitis. Similarly, other eye diseases, such as, for example, infections, diabetes, and uveitis, may also require multiple injections into the eye of a subject. By using the described methods and devices the requirement for multiple injections can be reduced or eliminated, while optionally using the same or similar therapeutic agents.

For example, to treat a patient suffering from wet age-related macular degeneration, an example drug held in the reservoir 125 can optionally be ranibizumab (Leucentis®) (Genentech Inc., San Francisco, Calif.) or benvacizumab (Avastin®) (Roche, Basel, Switzerland). Moreover, if the device comprises multiple reservoirs each reservoir can contain a different drug.

Other example, therapeutic agents or drug that can be used with the described methods and devices include VEGF inhibitors, including, for example anti-VEGF agents, siRNA, and agents that down regulate a VEGF membrane receptor. In addition, therapeutic agents may include nucleotide and polynucleotide sequences and/or vectors comprising the same. These sequences can encode expression of proteins or polypeptides effective for the treatment of eye diseases. The devices and method can therefore be used to deliver a variety of compositions to the eye and its surrounding tissues. The compositions can optionally treat eye disease in the eye to which the composition was delivered.

The described therapeutic agents can be located in one or more reservoir and released to treat a disease of the eye. For example, any eye disease can be treated that can be treated by administration of a therapeutic agent to the eye and its surrounding tissue. Some non-limiting diseases that can be treated include wet macular degeneration, inflammation, infection, diabetic pathologies, uveitis, Leber Congenital Amarosis, retinitis pigmentosa and tapetoretinal degenerations.

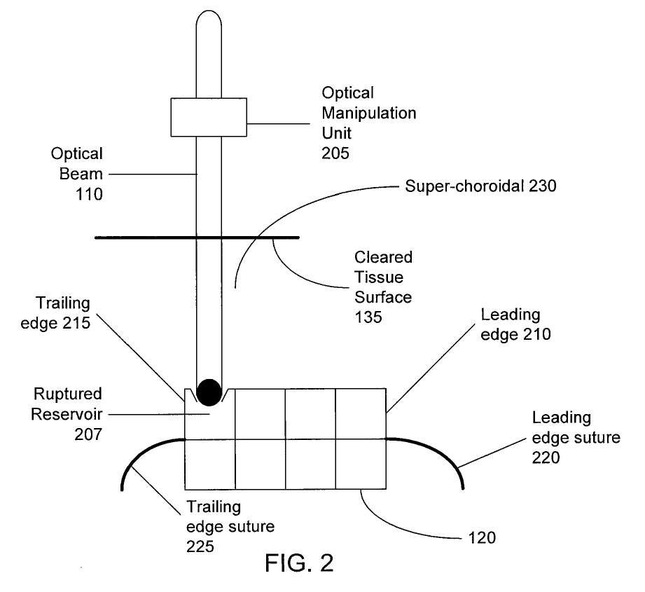

To permit the optical beam 110, for example, the laser beam generated by the Q-switched Nd-YAG laser, to pass through the surface of the eye tissue 115, the region of the surface of the eye can be treated to produce cleared tissue surface 135 that is at least partially transparent to light. In some implementations, a hyper-osmotic tissue clearing agent, for example 100% anhydrous glycerol, is applied to the eye surface thereby rendering the tissue surface of the eye more transparent to light, thereby permitting the laser beam from the Q-switched Nd-YAG laser or similar laser to pass through and impinge upon a reservoir 125 of the delivery device 120. The power of the laser beam is sufficient to rupture the reservoir 125. The reservoir 125 ruptures, the drug is released, and the released drug spreads through the diseased region of the eye through diffusion. The Q-switched Nd-YAG laser is switched off thereby cutting off the laser beam. Once the effect of the tissue clearing agent wears off, the eye becomes more opaque once again. These steps can be repeated at periodic intervals to release the drug stored in each of the reservoirs 125, as described with reference to FIG. 2.

FIG. 2 is a schematic illustration showing an example method to deliver a drug. The drug delivery device can be manufactured to include multiple reservoirs, each of which is configured to store the drug. Dimensions of the device and the included reservoirs are described with reference to FIG. 3. In some implementations, each reservoir can be manufactured to store micro-liters of the drug, for example, up to 7 μl or more at concentrations determined to treat wet age-related macular degeneration. For example, the reservoirs can be positioned adjacent to each other to form a layer of reservoirs. Further, the layers can be stacked on top of each other. In this manner, a volume of the drug in each reservoir can correspond to a recommended dosage of the drug. The device 120 can be sealed by sealing the multiple reservoirs. Each reservoir can be configured to rupture upon application of energy, for example, energy from a laser beam generated by a Q-switched Nd-YAG laser.

The delivery device 120 includes a leading edge suture 220 and a trailing edge suture 225 attached to a leading edge 210 and a trailing edge 215, respectively, of the device 120. The device 120 can be implanted underneath the eye tissue 115, for example, in the super-choroidal space 230, by securing the leading edge suture 220 and the trailing edge suture of the device 120 once the device 120 is positioned in or proximate the diseased region of the eye. In some implementations, an optical manipulation unit 205, including one or more optical components, for example, mirrors, lenses, and the like, can be used to focus the optical beam 110 on a particular or selected reservoir so that the reservoir ruptures. The drug in the ruptured reservoir 207 is released representing the release of one dosage of the drug to treat the diseased eye. Subsequently, using the optical manipulation unit 205, the optical beam 110 can be positioned on another reservoir, thereby rupturing the reservoir causing another release of the stored drug. In this manner, the multiple reservoirs can be ruptured. Once all reservoirs have been ruptured, the device 120 can be removed and optionally replaced with a new device. Example manufacturing specifications of a device 120 are described with reference to FIG. 3.

FIG. 3 shows an example of an apparatus 305 to deliver a drug. The apparatus 305 is manufactured using a biocompatible material, for example, ultraviolet-cured biocompatible polyurethane, such as polymethyl methacrylate (PMMA), polydimethylsiloxane (PDMS), and the like, or any other biocompatible material that is easy to manipulate during manufacturing. In some implementations, the PDMS can be cast on a mold having the inverse shape of the apparatus 305. Alternatively, or in addition, reservoirs can be etched in a biocompatible material using known techniques, such as, using hydrofluoric acid. In addition, the material is selected so that the material easily ruptures upon the application of energy, for example, when a pulsed laser beam emitted by a describe laser such as a Q-switched Nd-YAG laser is applied to the material. In some implementations, each of the reservoirs has a cuboid shape, i.e., each reservoir has six rectangular surfaces. In such implementations, two vertical opposing side walls of a reservoir are 2.6 mm apart. The walls of the reservoir adjacent to the leading or trailing edge of the apparatus 305 can be 0.3 mm from the leading or trailing edge. The top surface of the apparatus 305 can be a seal 310 that is 0.01 mm thick such that the apparatus 305 is 1.501 mm in height.

In some implementations, the seal 310 is opaque and is coated with titanium oxide, for example, 1.8 mg of TiO₂ per 1.0 g of PDMS, to improve the reflective properties of the apparatus 305. In some implementations, the entire apparatus 305 is 6 mm wide. In alternative implementations, both the dimensions of an apparatus and the shape of the reservoir can be different from those of apparatus 305. In general, the dimensions of each reservoir can depend on the dosage of the drug that is used to treat the diseased region of the eye. For example, the shape of each reservoir can be spherical, as shown in FIG. 4.

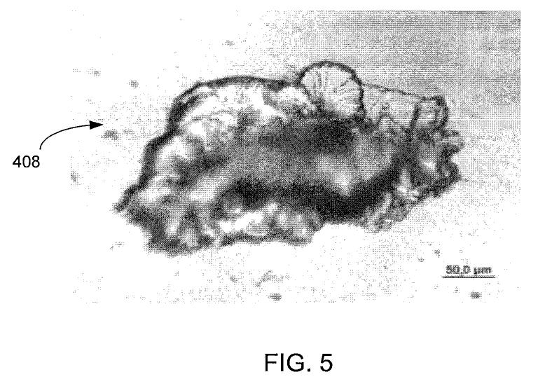

FIG. 4 shows an example of a drug delivery device 405. The device includes two 2.6 mm diameter reservoirs 406 including 1% sodium (Na) fluorescence dye. The reservoirs were filled with the dye and sealed with seal 408 under oxygen plasma. Four such devices 405 were implanted in the super-choiroidal spaces of rabbits' eyes by making a 4.0 mm incision at the sclera (0.5 mm from the cornea) such that the seal was facing toward the exterior of the eye. Using two surgical 10-1 sutures with 0.025 mm diameter, the ends of the devices were fastened to prevent any movement. This implant surgery was performed under full anesthesia. Once the rabbits' eyes recovered from the surgery, the respective devices 405 were checked for leaks. To do so, the pupil of a rabbit's eye was dilated with 3 drops of 5% homatropine every 1-1.5 minutes for 5 minutes. Once the pupil dilated, a linear fiber optic based probe of a noninvasive spectral diagnostic system was used to measure the intensity of 1% Na fluorescence in the aqueous and vitreous. In the spectral system, two light sources, namely, a pulsed xenon flash lamp to collect which light reflectance in the 350 nm-800 nm range, and a pulsed nitrogen laser to excite NADH and collagen fluorescence were used. Through this method, it was determined that the devices did not leak. The ability of the laser to rupture a reservoir in the device was tested as described with reference to FIG. 5.

FIG. 5 shows an example of a reservoir ruptured using an optical beam. To rupture the membrane or seal 408 covering a reservoir, an ophthalmic portable, class 3B, Q-switched Nd-YAG laser having 1064 nm wavelength was used. The laser had a Q-switch with a maximum power of 12.0 mJ. The beam profile was a 5 μm beam diameter. In an exemplary application, using a hypodermic needle, 1.0 mL of hyper-osmotic agent such as 100% anhydrous glycerol can be injected into the sclera over the region where the drug delivery device is implanted. The sclera becomes optically clear and stays clear for a period of time. Once the sclera becomes optically clear, the device becomes visible from the outer most layer of the eye, the conjuctiva. By applying a laser beam from the Q-switched Nd-YAG laser on the seal of a reservoir of the apparatus, the seal/membrane was ruptured, as shown in FIG. 5.

While this specification contains many specifics, these should not be construed as limitations on the scope of the specification or of what may be claimed, but rather as descriptions of features specific to particular implementations of the specification. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Thus, particular implementations of the specification have been described. Other implementations are within the scope of the following claims. For example, the actions recited in the claims can be performed in a different order and still achieve desirable results. In some implementations, the half life of the fluorescent dye and the path taken by the dye as it diffuses through the eye can be tracked, for example using a non-invasive spectral diagnostic system. The fluorescence measurement can be done 1 hour after the initial release and can be performed every three days up to 15 days to get the decay of the fluorescence dye in the vitreous humor of the eye.

What is claimed is:

1. A method for treating an eye disease, comprising:
a. implanting a device comprising a sealed container into a diseased eye, wherein a dosage of drug for treating the diseased eye is disposed within the sealed container; and
b. applying one or more pulses of light energy onto the sealed container wherein the one or more pulses causes the selective release the dosage of drug from the sealed container into eye tissue, wherein the released dosage treats the eye disease.

2. The method of claim 1, wherein the device is implanted under the sclera of the eye.

3. The method of claim 1, wherein the device is implanted in the superchorodial space of the eye.

4. The method of claim 1, wherein the device is implanted in the posterior chamber of the eye.

5. The method of claim 1, wherein the device is implanted in the sclera of the eye.

6. The method of claim 1, wherein the device is implanted episclerally in the eye.

7. The method of claim 1, wherein the one or more pulses of light energy are directed onto the sealed container of the implanted device, the one or more light pulses causing rupture of at least a portion of the sealed chamber such that the drug dosage is selectively released from the chamber into eye tissue.

8. The method of claim 7, wherein the one or more pulses of light creates a shock wave that disrupts at least a portion of the sealed chamber.

9. The method of claim 7, wherein the one or more pulses of light do not substantially heat the sealed chamber or a portion thereof to cause rupture of the sealed chamber.

10. The method of claim 7, wherein the one or more pulses of light are directed through the sclera and then onto the sealed container of the implanted device, the light causing rupture of at least a portion of the sealed chamber such that the drug dosage is selectively released from the chamber into eye tissue.

11. The method of claim 10, further comprising treating the sclera with an agent that alters the optical properties of the sclera to allow increased transmission of light through the sclera, wherein the scleral treatment occurs prior to directing the light through the sclera and onto the sealed container of the implanted device.

12. The method of claim 1, wherein the disease is selected from the group consisting of wet age-related macular degeneration, infection, diabetes and uveitis.

13. The method of claim 1, wherein the implanted device comprises a plurality of sealed containers wherein a dosage of drug for treating the diseased eye is disposed within each sealed container.

14. The method of claim 13, wherein each sealed container of the implanted device is configured to be individually ruptured to allow drug from the individually ruptured sealed container to enter the eye tissue.

15. The method of claim 14, wherein a first sealed container is ruptured and a second sealed container is ruptured at a predetermined time subsequent to the rupture of the first sealed container.

16. A method for delivering a drug to an eye lesion, the method comprising:
treating the eye with an agent configured to render a region of the eye at least partially transparent to light or more transparent to light,
positioning a drug delivery device including one or more reservoirs in proximity to the eye lesion, each reservoir including the drug; and
rupturing one or more the reservoirs by applying light generated from a light source through the region rendered partially or more transparent to light and onto the drug delivery device, the rupturing of the one or more reservoirs causing delivery of the drug to the eye lesion.

17. The method of claim 16, further comprising:
generating one or more light pulses from a laser source; and
manipulating at least one light pulse to be incident on a reservoir including the drug, wherein the pulse ruptures the reservoir on which the pulse is incident.

18. An apparatus to deliver a drug, the apparatus comprising:
a device including at least one reservoir;
a drug to be delivered, a dosage of the drug included in the reservoir; and
a seal sealing the reservoir, the seal comprising a material which ruptures without melting when an optical pulse from a laser source is directed onto the seal.

19. The apparatus of claim 18, wherein the device is an ocular implant configured for positioning beneath the sclera of the eye to be treated.

20. The apparatus of claim 18, wherein the device is an ocular implant configured for positioning in the superchorodial space of the eye to be treated.

21. The apparatus of claim 18, wherein the device is an ocular implant configured for positioning in the posterior chamber of the eye to be treated.

22. The apparatus of claim 18, wherein the device is an ocular implant configured for positioning in the sclera of the eye.

23. The apparatus of claim 18, wherein the device is an ocular implant configured for positioning episclerally in the eye.
